(19) United States
(12) Patent Application Publication
(10) Pub. No.: US 2023/0338684 A1
(43) Pub. Date: Oct. 26, 2023

(54) TRACHEOSTOMY TUBE SPEAKING VALVE

(71) Applicant: Brian Kamradt, Martinsville, IN (US)

(72) Inventor: Brian Kamradt, Martinsville, IN (US)

(21) Appl. No.: 18/137,476

(22) Filed: Apr. 21, 2023

Related U.S. Application Data
(63) Continuation of application No. 63/333,344, filed on Apr. 21, 2022.

(51) Int. Cl. A61M 16/04

(52) U.S. Cl. CPC A61M 16/0468

(57) ABSTRACT

A tracheostomy tube speaking valve is disclosed. The tracheostomy tube speaking valve includes a cylindrical body with a passageway for air to pass through. A valve disc is provided in the passageway. The disc is moveable to allow for inhalation through the passageway and will close automatically in response to exhalation pressure to direct air to the patient's larynx and mouth for speech.

### PRIORITY

The present application is related to, and claims the priority benefit of, U.S. Provisional Patent Application Ser. No. 63/333,344, filed Apr. 21, 2022, the contents of which are incorporated herein directly and by reference in their entirety.

### TECHNICAL FIELD

This present disclosure relates to speech aids for tracheotomized individuals.

### BACKGROUND

Patients suffering from chronic obstructive pulmonary diseases and neuro-muscular diseases often require support ventilation while sleeping in order to prevent the occurrence of carbon dioxide narcosis. This type of ventilation generally requires the installation of a tracheostomy tube. Because the tracheostomy tube remains in place during the day, it is generally desirable to attach a one-way, or speaking valve, to the outward end of the tracheostomy tube. The valve allows the patient to carry on conversations.

Patients complain that the speaking valves are uncomfortable because of the initial effort required to bias the valve open at the beginning of each inhalation. This increased effort can exhaust the patient. Mucus contamination of the valve can significantly increase the effort required to bias the valve open.

One such speaking valve consists of a flexible disc-shaped diaphragm that collapses inward upon inhalation and seals against a fixed surface upon exhalation. There are two variations of this type valve. In the first variation, of which the description in U.S. Pat. No. 3,137,299 to Tabor is exemplary, the disc-shaped diaphragm is attached to the valve housing through its center. In the second variation, the disc shaped diaphragm is attached to the valve housing along one edge. U.S. Pat. No. 4,040,428 to Clifford is exemplary of this variation. Another variation uses a ball valve that is placed with in the Tracheostomy tube thus restricting airflow making it uncomfortable to operate and exhaust the patient. U.S. Pat. No. 6,588,428B2

Valve sticking can become a problem with these types of valves because, as mucus accumulates, the flexible diaphragm can become tacky and begin to adhere to the fixed surface during each exhalation. Since the diaphragm is now adhering to the fixed surface, the patient must expend more force inhaling and exhaling to create a vacuum in the conduit of the valve housing which is sufficient to free the edge of the diaphragm from the fixed surface. This can become very uncomfortable after even a short period of time. It would be desirable, therefore, to have a tracheostomy valve which would require less inhalation effort to bias open even when the seal becomes tacky.

### BRIEF SUMMARY

Therefore, it is an object of the present disclosure to provide a tracheostomy tube valve which will allow the patient to talk but which requires very little force differential to bias into the open or closed position.

It is a further object of the present disclosure to provide a tracheostomy tube valve which is not affixed to any post or rib and thus able to move uninhibited.

Accordingly, a tracheostomy tube valve for use in conjunction with a tracheostomy tube is described. The valve unit comprises a valve body having a first end, a second end and a fluid passageway extending therethrough. The first end is configured to be operably connected with the tracheostomy tube. A disc is disposed within the fluid passageway spaced between the first end of the valve body and the retaining means. Seating means for the disc is disposed at the first open end of the valve body. Guiding means are formed within the valve body to maintain the disc substantially centered wherein air flow about the disc is unrestricted. When the patient exhales, due to the unrestricted air flow, minimum force is required to move the disc against the seating means to seal the opening in the first end of the valve body and prevent exhaled air from exiting thus forcing air through the larynx, and when the patient inhales, the disc is moved away from the seating means in the first end of the tube and retained by the retaining means wherein air enters the tracheostomy tube.

The present disclosure includes disclosure of a tracheostomy apparatus, comprising a curved cannula having an open first end and an opposite second end configured for surgical insertion into a trachea of a patient, a neck plate connected to the curved cannula and configured for disposal on an outside of a throat of the patient, and a valve comprising a hollow cylindrical body configured to fit over the curved cannula, the valve comprising at least three circumferentially spaced-apart longitudinal ribs formed on an inner surface of the hollow cylindrical body.

The present disclosure includes disclosure of a tracheostomy apparatus, wherein the curved cannula is bent at or about 90 degrees.

The present disclosure includes disclosure of a tracheostomy apparatus, wherein when used by the patient, the open first end extends outwardly from a throat of the patient, and the opposite second end extends downwardly toward lungs of the patient.

The present disclosure includes disclosure of a tracheostomy apparatus, wherein the valve further comprises retaining means disposed within the hollow cylindrical body.

The present disclosure includes disclosure of a tracheostomy apparatus, wherein the retaining means comprises a series of ribs perpendicular to and positioned through the hollow cylindrical body.

The present disclosure includes disclosure of a tracheostomy apparatus, further comprising a disc disposed within the hollow cylindrical body between a proximal opening and the retaining means.

The present disclosure includes disclosure of a tracheostomy apparatus, wherein an annular flat surface is formed within the hollow cylindrical body so that the disc is seated when the patient exhales.

The present disclosure includes disclosure of a tracheostomy apparatus, wherein when the disc is seated, a seal is created, preventing exhaled air from passing through the tracheostomy tube.

The present disclosure includes disclosure of a tracheostomy apparatus, wherein when used by the patient, inhaled air passes through the valve, around the disc, and into the curved cannula.

The present disclosure includes disclosure of a tracheostomy apparatus, wherein when used by the patient, exhaled air passed through the curved cannula to the disc, causing the disc to be seated, creating a seal.

The present disclosure includes disclosure of a tracheostomy apparatus, whereby the exhaled air is forced to enter a larynx of the patient, improving speech quality of the patient when the patent attempts to speak.

The present disclosure includes disclosure of a valve apparatus, comprising a hollow cylindrical body configured to fit over a curved cannula, the valve comprising at least three circumferentially spaced-apart longitudinal ribs formed on an inner surface of the hollow cylindrical body, retaining means disposed within the hollow cylindrical body, the retaining means comprising a series of ribs perpendicular to and positioned through the hollow cylindrical body, and a disc disposed within the hollow cylindrical body between a proximal opening and the retaining means.

The present disclosure includes disclosure of a valve apparatus, comprising part of a tracheostomy apparatus, the tracheostomy apparatus further comprising a curved cannula having an open first end and an opposite second end configured for surgical insertion into a trachea of a patient, and a neck plate connected to the curved cannula and configured for disposal on an outside of throat of the patient.

The present disclosure includes disclosure of a valve apparatus, wherein when the tracheostomy apparatus is used by the patient, inhaled air can pass through the proximal opening of the valve apparatus, around the disc, into the curved cannula, and into lungs of the patient.

The present disclosure includes disclosure of a valve apparatus, wherein when the tracheostomy apparatus is used by the patient, exhaled air causes the disc to form a seal within the valve apparatus, effectively diverting the exhaled air through a larynx of the patient, improving speech quality of the patient when the patent attempts to speak.

The present disclosure includes disclosure of a method of improving speech quality, comprising the steps of positioning the opposite second end of the curved cannula of the tracheostomy apparatus of claim 1 into the trachea of the patient, and positioning the neck plate on a relative outside of a neck of the patient, wherein inhaled air can pass through the valve into lungs of the patient, and wherein exhaled air causes the valve to close, diverting the exhaled air into a larynx of the patient, improving speech quality of the patient when the patent attempts to speak.

The present disclosure includes disclosure of a method of improving speech quality, wherein the valve is configured to close due to a disc positioned within the valve being able to seal an opening of the valve.

Further advantageous features of the present disclosure are referenced herein.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a perspective view of a valve, according to an exemplary embodiment of the present disclosure;

FIG. 2 shows a perspective view of a tracheostomy tube with the valve of the present invention fitted to the proximal 15 mm end of the tube, according to an exemplary embodiment of the present disclosure;

FIG. 3 shows a longitudinal-section view taken across the lines A-A of FIG. 1, according to an exemplary embodiment of the present disclosure;

FIG. 4 shows a cross-section view thereof taken across the lines B-B of FIG. 3, according to an exemplary embodiment of the present disclosure;

FIG. 5 shows an exploded view of an exemplary device, showing it pieces, according to an exemplary embodiment of the present disclosure;

FIGS. 6-9 show a sequence of diagrams showing the operation of exemplary devices of the present disclosure, whereby:

FIG. 6 shows the disc in at a rest position within the cylindrical valve body, according to an exemplary embodiment of the present disclosure;

FIG. 7 shows the disc held at the retaining means during inhalation with air flow around the disc, according to an exemplary embodiment of the present disclosure;

FIG. 8 shows the disc moving towards the proximal opening during exhalation, according to an exemplary embodiment of the present disclosure; and

FIG. 9 shows the disc seated on the seating means adjoining the proximal opening creating a seal so that exhalation air must divert and pass into the larynx during expiration, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

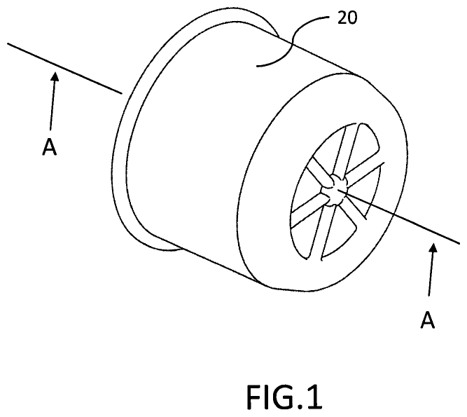

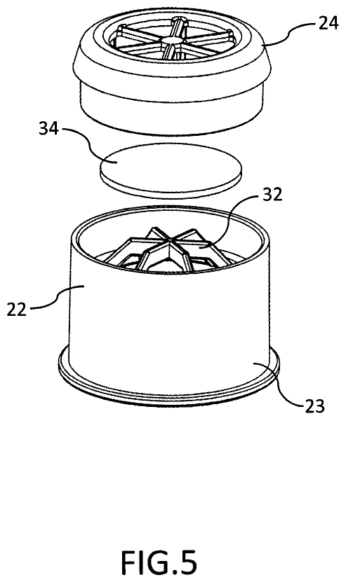

Referring now to FIGS. 1-9, a tracheostomy tube 10 is shown, comprising a cannula 12 having an open first end 13 configured to extend outwardly from the throat of a patient, and a second end 14 which is configured to be surgically inserted into the trachea of the patient. The second end 14 is then directed downwardly toward the lungs of the patient with the tracheostomy tube 10 being below the larynx. A neck plate 15 is connected to the outside of the cannula 12 and is disposed on the outside of the patient's throat to stabilize the cannula 12 within the surgical opening in the throat.

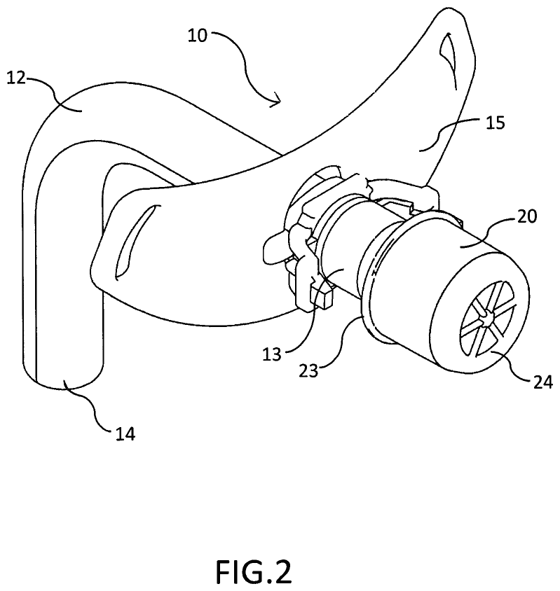

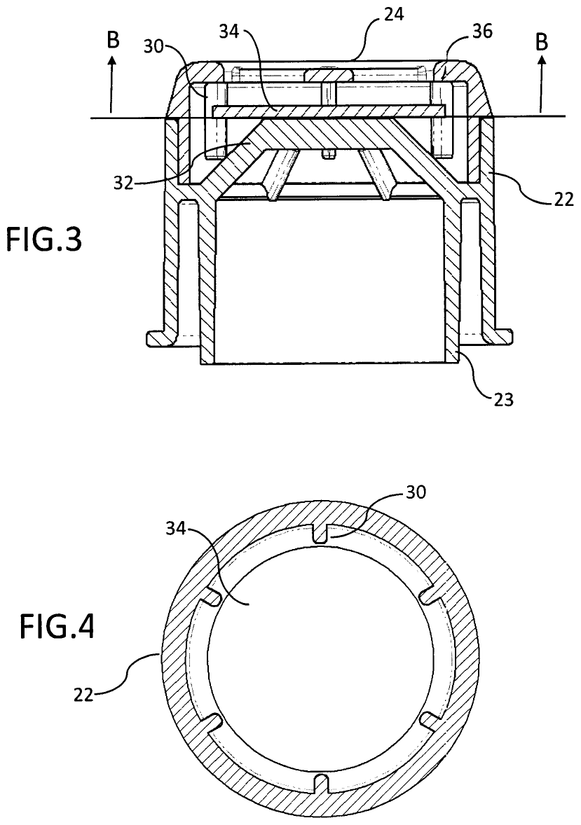

FIG. 1 shows an exemplary valve 20 of the present disclosure. FIG. 2 shows the valve 20 fitted over the standard 15 mm cannula 12. The valve 20, as shown in FIGS. 2 and 3, has a hollow cylindrical body 22 with both a distal end 23 and a proximal end 24 and being open at both ends 23, 24 with the distal end 23 having an inner diameter so to be snugly fitted (with a friction fit) over the standard 15 mm end 13 of the tracheostomy tube 10.

FIGS. 3 and 4 show a plurality of guiding means 30 that are formed within the cylindrical body 22 at the proximal end 24. Preferably, the guiding means 30 are at least three circumferentially spaced-apart longitudinal ribs formed on the inner surface of the hollow cylindrical body 22. The guiding means 30 (also referred to herein as ribs 30) are very thin to have minimum effect on air flow through the body, whereby a thickness of approximately 0.01 inches is preferred. Retaining means 32 are disposed within the cylindrical body 22 proximal end 24. The ribs or guiding means 30 have a length extending from the proximal opening 26 to the retaining means 32. The retaining means 32 is a series of ribs perpendicular to and placed through the body 22. A disc 34 is disposed within the body 22 between the proximal opening 24 and the retaining means 32 and intermediate of the guiding means 30. The disc 34 is substantially centered within the cylindrical body 22. An annular flat surface 36 is formed within the body 22 adjoining the proximal opening 26 and serves as a seating means (annular flat surface 36) for the disc 34 when the patient exhales. When the disc 34 is seated on the flat surface 36, a seal 38 is created, and exhaled air is prevented from passing through the tracheostomy tube 10 but is forced up the trachea passing the larynx.

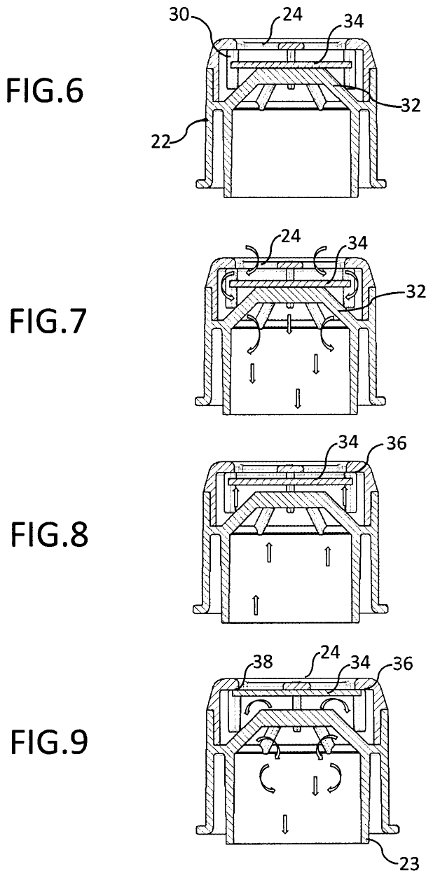

FIGS. 6-9 show the operation of tracheostomy tubes 10 of the present disclosure. At rest (FIG. 6) the disc 34 is intermediate of the guiding means 30 between the proximal opening 26 of body 22 and the retaining means 32. When the patient inhales (FIG. 7), the air incoming through the opening 26 moves the disc 34 against the retaining means 32 and air flows in an unrestricted manner into the lungs of the patient. When the patient exhales (FIG. 8), minimal force of the initial breath pushes the disc 34 up towards the seating means 36. (FIG. 9) minimal force has pushed the disc 34 against the seating means 36 on the inner face of the proximal opening 26 creating a seal 38. Due to the configuration of devices of the present disclosure, the exhaled air cannot pass through the valve 20 and is forced to pass through the tracheostomy tube 10 and enter the larynx improving the quality of speech of the patient.

A disc valve 20 to control inspiration and expiration of air must consider flow rates through the valve 20. Anytime there is a restriction of a smaller cross-sectional area, an interface is created which changes the opposition presented to air flow. Ideally, the cross-sectional area presented to the movement of air should be constant and not be restricted within the cannula. A ball valve that is inserted into the tracheostomy tube cannula (such as described in U.S. Pat. No. 6,588,428) is restrictive and creates a smaller cross-sectional area and changes the opposition presented to air flow. To approach a constant cross-sectional area, a spherical chamber that extends outside the cannula 12 is suggested to house a disc valve 20.

The difference between the cross-sectional area of the chamber and that of the disc 34 is equal to the cross-sectional area of the cannula 12 so that there are no restrictions and, hence, air flow is at a constant velocity. As a consequence, the force required to inhale and exhale is reduced and there is reduced burden on the patient. A further consequence is that the speech valve 20 in the cannula 12 closes more rapidly on inhalation due to the reduced force required and the vocal cords are activated almost immediately when the patient speaks.

While various embodiments of devices and methods have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A tracheostomy apparatus, comprising:
a curved cannula having an open first end and an opposite second end configured for surgical insertion into a trachea of a patient;
a neck plate connected to the curved cannula and configured for disposal on an outside of a throat of the patient; and
a valve comprising a hollow cylindrical body configured to fit over the curved cannula, the valve comprising at least three circumferentially spaced-apart longitudinal ribs formed on an inner surface of the hollow cylindrical body.

2. The tracheostomy apparatus of claim 1, wherein the curved cannula is bent at or about 90 degrees.

3. The tracheostomy apparatus of claim 1, wherein when used by the patient, the open first end extends outwardly from a throat of the patient, and the opposite second end extends downwardly toward lungs of the patient.

4. The tracheostomy apparatus of claim 1, wherein the valve further comprises retaining means disposed within the hollow cylindrical body.

5. The tracheostomy apparatus of claim 4, wherein the retaining means comprises a series of ribs perpendicular to and positioned through the hollow cylindrical body.

6. The tracheostomy apparatus of claim 4, further comprising a disc disposed within the hollow cylindrical body between a proximal opening and the retaining means.

7. The tracheostomy apparatus of claim 6, wherein an annular flat surface is formed within the hollow cylindrical body so that the disc is seated when the patient exhales.

8. The tracheostomy apparatus of claim 7, wherein when the disc is seated, a seal is created, preventing exhaled air from passing through the tracheostomy tube.

9. The tracheostomy apparatus of claim 6, wherein when used by the patient, inhaled air passes through the valve, around the disc, and into the curved cannula.

10. The tracheostomy apparatus of claim 4, wherein when used by the patient, exhaled air passed through the curved cannula to the disc, causing the disc to be seated, creating a seal.

11. The tracheostomy apparatus of claim 10, whereby the exhaled air is forced to enter a larynx of the patient, improving speech quality of the patient when the patent attempts to speak.

12. A valve apparatus, comprising:
a hollow cylindrical body configured to fit over a curved cannula, the valve comprising at least three circumferentially spaced-apart longitudinal ribs formed on an inner surface of the hollow cylindrical body;
retaining means disposed within the hollow cylindrical body, the retaining means comprising a series of ribs perpendicular to and positioned through the hollow cylindrical body; and
a disc disposed within the hollow cylindrical body between a proximal opening and the retaining means.

13. The valve apparatus of claim 12, comprising part of a tracheostomy apparatus, the tracheostomy apparatus further comprising:
a curved cannula having an open first end and an opposite second end configured for surgical insertion into a trachea of a patient; and
a neck plate connected to the curved cannula and configured for disposal on an outside of throat of the patient.

14. The valve apparatus of claim 12, wherein when the tracheostomy apparatus is used by the patient, inhaled air can pass through the proximal opening of the valve apparatus, around the disc, into the curved cannula, and into lungs of the patient.

15. The valve apparatus of claim 12, wherein when the tracheostomy apparatus is used by the patient, exhaled air causes the disc to form a seal within the valve apparatus, effectively diverting the exhaled air through a larynx of the patient, improving speech quality of the patient when the patent attempts to speak.

16. A method of improving speech quality, comprising the steps of:
positioning the opposite second end of the curved cannula of the tracheostomy apparatus of claim 1 into the trachea of the patient; and
positioning the neck plate on a relative outside of a neck of the patient;
wherein inhaled air can pass through the valve into lungs of the patient; and
wherein exhaled air causes the valve to close, diverting the exhaled air into a larynx of the patient, improving speech quality of the patient when the patent attempts to speak.

17. The method of claim 16, wherein the valve is configured to close due to a disc positioned within the valve being able to seal an opening of the valve.
